(19) United States
(12) Patent Application Publication
(10) Pub. No.: US 2024/0359033 A1
(43) Pub. Date: Oct. 31, 2024

(54) LASER ENERGY MONITOR FOR MYOPIA TREATMENT DEVICE AND MONITORING METHOD

(71) Applicant: Guanglang (Hainan) Biotechnology Co., Ltd., Haikou City (CN)

(72) Inventor: Te GAO, Haikou City (CN)

(21) Appl. No.: 18/476,709

(22) Filed: Sep. 28, 2023

(30) Foreign Application Priority Data
Apr. 28, 2023 (CN) 202310485448.6

(51) Int. Cl. A61N 5/067

(52) U.S. Cl. CPC A61N 5/067; A61N 2005/0626; A61N 2005/0664; A61N 2005/0665

(57) ABSTRACT

The present disclosure belongs to the technical field of laser energy monitoring, and particularly, a laser energy monitor for myopia treatment device and a monitoring method are provided. The monitor comprises a laser light source, a housing, a beam shaping lens, a beam homogenizing lens, a photosensitive sensor, and a controller; wherein the housing is provided with a cavity for receiving the laser light source, beam shaping lens, beam homogenizing lens, and photosensitive sensor, the housing is also provided with a light aperture, the light aperture is communicated to the cavity, and the laser light source is positioned opposite to the light aperture; and the photosensitive sensor is electrically connected to the controller. The monitor can adjust the inner layout of the conventional housing.

### CROSS-REFERENCE TO RELATED APPLICATIONS

This application claims priority is based on Chinese Application No. 202310485448.6, having a filing date of Apr. 28, 2023, the entire contents of which are hereby incorporated by reference.

### FIELD OF TECHNOLOGY

The following belongs to the technical field of laser energy monitoring, and particularly, it relates to a laser energy monitor for myopia treatment device and a monitoring method.

### BACKGROUND

As technology is developing, the categories of electronic devices are gradually increasing. Users' long-term usages of electronic devices with screens often lead to symptoms, such as eye fatigue or even myopia, resulting in an increasing proportion of myopia year by year. Therefore, the daily prevention and treatment of myopia and myopia control have become a new development trend. Some equipment can be used for exercising eyes in vision therapy in near/far vision field, and can also be used for simulating some light to irradiate eyeballs in irradiation therapy, which plays a certain role in the prevention and treatment of myopia. Generally, low-level laser light is used to treat the eyes in photobiomodulation therapy (PBMT), and the control of laser is particularly important to the therapy. Repeated low-level read-light (RLRL) therapy, which is one of PBMT, has been used into practice on childhood myopia control. An appropriate laser light irradiation can achieve a good therapeutic effect. For example, a device emits low-level high-brightness red light with a specific wavelength of 650 nm to irradiate the eyeballs. However, high-level laser light can injure the eyes and cannot achieve the purpose of treatment. Therefore, when performing a laser irradiation therapy with equipment, laser intensity monitoring is particularly important.

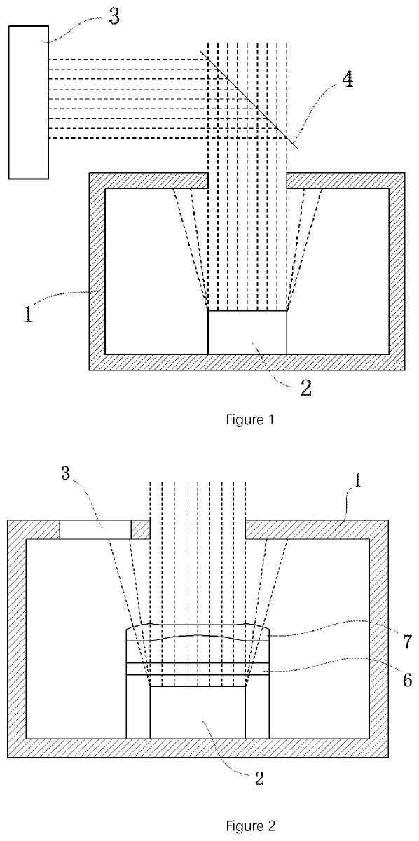

In the conventional art, the intensity of the light source is measured and calculated by using the method of reflecting light beam from a semi-transparent and semi-reflective mirror. This method is implemented as FIG. 1, wherein a laser light source is configured inside a housing of the light source, the housing is provided with a light aperture, which is capable of blocking a marginal ray from the light beam emitted by the laser light source and only retaining a chief ray in the middle. The chief ray in the middle is basically emitted outwardly in the emitting direction of the laser light source. When the light beam is emitted to the semi-transparent and semi-reflective mirror 4 outside of the housing, a portion of the light beam is reflected, and another portion of the light beam continues to be emitted forward through the semi-transparent and semi-reflective mirror. As the light transmission of the semi-transparent and semi-reflective mirror is calibrated, it is generally available to obtain a semi-transparent and semi-reflective mirror with a desired light transmission in market. For example, the light transmission is 50%, indicating that half of the light beam passes through the semi-transparent and semi-reflective mirror, while the other half is reflected.

The light beam reflected by the semi-transparent and semi-reflective mirror is emitted to a photosensitive sensor, which is configured adjacently to the semi-transparent and semi-reflective mirror. The photosensitive sensor is electrically connected to a control module and transmits detected light beam data to the control module. Finally, the control module obtains the intensity of the light beam that passes through the semi-transparent and semi-reflective mirror by calculation. Users can adjust the laser light source according to the calculated light intensity, and then adjust the light intensity of the laser light beam passing through the semi-transparent and semi-reflective mirror, to obtain the optimal light intensity for usage.

However, such design has a complicate structure and a large size, and the reflection of the laser light source is also difficult to adjust, leading to a high manufacturing cost for the equipment. In addition, the light spot emitted by the light from the laser light source is elliptical, and its energy distribution is not uniform. Specifically, the central energy is high, while the marginal energy is low. Therefore, it has a certain impact on the accuracy of the detection results, resulting in a large error in the intensity of the light beam detected by the monitor.

Thus, for one skilled in the art, it is an urgent demand to obtain a laser energy monitor, with simple structure, small monitoring error, low manufacturing difficulty and low manufacturing cost.

### SUMMARY

In order to overcome the above-mentioned defeats, an aspect relates to a laser energy monitor for myopia treatment device, comprising:
a laser light source, a housing, a beam shaping lens, a beam homogenizing lens, a photosensitive sensor, and a controller;
wherein the housing is provided with a cavity for receiving the laser light source, the beam shaping lens, the beam homogenizing lens, and the photosensitive sensor, the housing is also provided with a light aperture, the light aperture is communicated to the cavity, and the laser light source is positioned opposite to the light aperture;
the beam shaping lens and the beam homogenizing lens are configured between the laser light source and the light aperture, and the light aperture, the beam homogenizing lens, the beam shaping lens, and the laser light source are successively arranged from top to bottom; and the photosensitive sensor is electrically connected to the controller.

In one example, the photosensitive sensor is configured on an inner wall of the housing and adjacent to the light aperture.

In one example, the photosensitive sensor is configured on an inner wall of the housing and adjacent to the laser light source, and a reflector is configured on the inner wall of the housing and adjacent to the light aperture.

In some examples, the reflector is positioned opposite to the photosensitive sensor and adjacent to the light aperture, and a reflecting surface of the reflector is faced towards the photosensitive sensor.

In one example, the beam shaping lens is used for transforming a light beam emitted by the laser light source from an elliptical beam to a circular beam.

In one example, the beam shaping lens is provided with a beam input side and a beam output side; and the light beam emitted from the laser light source has a divergence angle from 10 to 45 degrees on the major-axis and a divergence angle from 0 to 30 degrees on the minor-axis when entering the beam input side of the beam shaping lens, and the light beam emitted from the laser light source have a divergence angle from 0 to 30 degrees on the both major-axis and minor-axis when exiting the beam output side of the beam shaping lens.

In one example, the laser light source is distanced from the beam input side of the beam shaping lens in a range from 1 to 10 mm, and the laser light source is distanced from the beam output side of the beam shaping lens in a range from 5 to 20 mm.

In one example, the beam homogenizing lens is an aspherical lens, configured for transforming the light beam that passes through the beam shaping lens into a flat-top distribution to obtain a circular collimated flat-top beam with a uniform intensity distribution.

Another aspect relates to a monitoring method for using in the laser energy monitor for myopia treatment device, and the method comprises the steps of:
Step 1. initiating a laser light source and emitting a light beam;
Step 2. receiving a portion of the light beam from the laser light source with a photosensitive sensor and obtaining a light intensity, then transmitting the light intensity to a controller;
Step 3. adjusting a brightness of the laser light source according to the detected light intensity from the controller.

In one example, in the Step 2, when the reflector is configured on an inner wall of the housing, the light intensity of the light beam at the light aperture detected by a sensor (not shown) is set as A, and the intensity of light beam detected by the photosensitive sensor is set as B, and the light beam attenuation coefficient (x) is calculated by comparing B with A. Therefore, a light beam attenuation compensation formula is obtained as follows: A=B*x. According to the light intensity detected by the photosensitive sensor (B) and the light beam attenuation coefficient (x), the light intensity of the light beam at the light aperture is calculated by such a compensation formula in real time.

Compared with the conventional art, the present disclosure has the advantages as follows:

The present disclosure provides a laser energy monitor for myopia treatment device and a monitoring method for using in this monitor. The monitor has adjusted the conventional inner layout of the conventional housing. Specifically, the beam shaping structure and the sensing structure are integrated inside the housing to reduce the size of the monitor. After the irradiation intensity of laser light in a unit area is calculated by the photosensitive sensor, the light intensity of the light beam at the light aperture is finally obtained. Therefore, the light intensity of the light beam at the light aperture can be obtained without complicate and fine adjustment.

### BRIEF DESCRIPTION

To more clearly explain the examples of the present disclosure, the accompanying drawings in the embodiments are briefly explained. Obviously, the drawings described below are some examples of the present disclosure. For one skilled in the art, other drawings can also be obtained based on these drawings without any creative work.

Some examples of the present disclosure will be described in detail, with reference to the following figures, wherein like designations denote like members, wherein:

FIG. 1 depicts a schematic diagram of the monitor in the conventional art;

FIG. 2 depicts a schematic diagram of a monitor in an embodiment of the present disclosure;

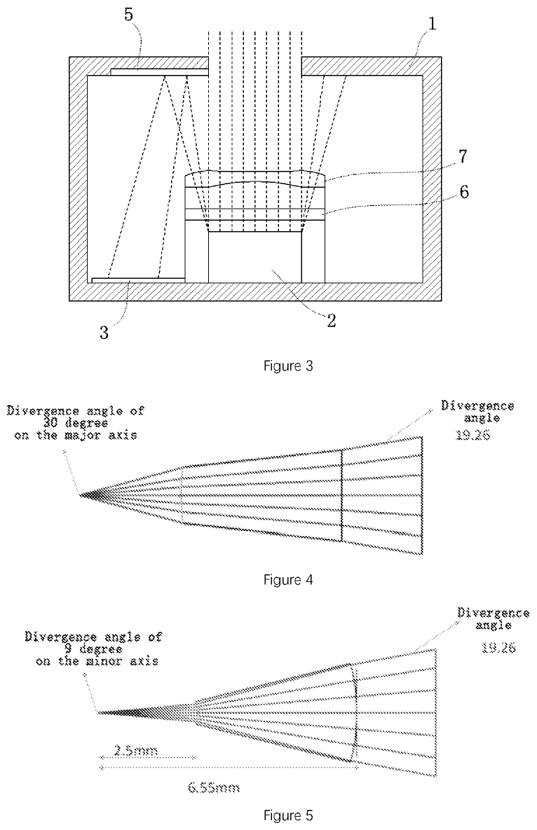

FIG. 3 depicts a schematic diagram of another monitor in an embodiment of the present disclosure;

FIG. 4 depicts a schematic diagram of the beam path adjustment of the light beam emitted from the laser light source on the major-axis at the beam input side of the light shaping lens; and

FIG. 5 depicts a schematic diagram of the beam path adjustment of the light beam emitted from the laser light source on the minor-axis at the beam input side of the light shaping lens;

wherein:
1—housing, 2—laser light source, 3—photosensitive sensor, 4—semi-transparent and semi—reflective mirror, 5—reflector, 6—beam shaping lens, 7—beam homogenizing lens.

### DETAILED DESCRIPTION

For a clearer understanding of the above purposes, features and advantages of the present disclosure, the present disclosure is described in detail below in conjunction with the accompanying drawings and specific embodiments. It is to be noted that the examples and the features in the embodiments of the present disclosure may be combined with each other without conflict. Many specific details are explained in the following description to facilitate a full understanding of the present disclosure, and the described examples are only a part of the embodiments of the present disclosure, but not all of the embodiments. Based on the examples of the present disclosure, all other embodiments, which are obtained by one skilled in the art without creative work, are within the protection scope of the present disclosure.

Unless otherwise defined, all technical and scientific terms used herein have the same meanings as generally understood by one skilled in the technical field to which the present disclosure belongs. The terms used herein in the specification of the present disclosure are only for the purpose of describing the specific embodiments and are not intended to limit the present disclosure.

### Example

As shown in FIGS. 2 to 5, the example of the present disclosure provides a laser energy monitor for myopia treatment device, comprising:
a laser light source 2, a housing 1, a beam shaping lens 6, a beam homogenizing lens 7, a photosensitive sensor 3 and a controller;
wherein the housing 1 is provided with a cavity for receiving the laser light source 2, the beam shaping lens 6, the beam homogenizing lens 7, and the photosensitive sensor 3, the housing 1 is also provided with a light aperture, the light aperture is communicated to the cavity, and the laser light source 2 is positioned opposite to the light aperture;
the beam shaping lens 6 and the beam homogenizing lens 7 are configured between the laser light source 2 and the light aperture, and the light aperture, the beam homogenizing lens 7, the beam shaping lens 6, and the laser light source 2 are successively arranged from top to bottom; and
the photosensitive sensor 3 is electrically connected to the controller.

The monitor works as follows:

After the light beam is emitted by the laser light source 2, the light beam firstly passes through the beam shaping lens 6. The divergence angles of the light beam on the major-axis and minor-axis are adjusted by the beam shaping lens 6 to obtain a nearly circular light spot (elliptical light spot), wherein the major-axis is the longest diameter of the elliptical light spot and the minor-axis is the shortest diameter of the elliptical light spot. Therefore, the spot on the inner wall of the housing 1 is closely circular.

The light intensity of the light beam at the margin of the light spot irradiated by the laser light source 2 is partially attenuated to some degree, compared with the light intensity of the light beam in the center of the light spot. Even though the light beam has been shaped, the light energy at the margin of the light spot is still smaller than that in the center of the light spot. Therefore, it is necessary to homogenize the energy of the light spot to improve the accuracy of detection. Based on this, a beam homogenizing lens 7 is provided. After the light beam passes through the shaping lens 6, it will also pass through the homogenizing lens 7, scattering the energy from the center to the margin of the light spot, and homogenizing the overall energy of the light spot. In such a way, it can be guaranteed that the intensity of the light beam passing through the light aperture is basically the same as that of the light beam irradiating on the inner wall of the housing 1. The provided photosensitive sensor 3 transmits the detected light intensity data to a controller that is electronically connected to the photosensitive sensor 3. As the intensity of the light beam irradiating on the photosensitive sensor 3 is basically the same as that of the light beam passing through the light aperture, the controller can obtain the intensity data of the light beam passing through the light aperture. When users adjust the brightness of laser light source 2, the change of light intensity is synchronously monitored by the photosensitive sensor 3, thereby adjusting the light intensity according to the real-time monitoring data. Finally, an appropriate intensity of the light beam is obtained.

In one example, as shown in FIG. 2, the photosensitive sensor 3 is configured on an inner wall of the housing 1 and adjacent to the light aperture.

In another example, as shown in FIG. 3, the photosensitive sensor 3 is configured on an inner wall of the housing and adjacent to the laser light source 2, and a reflector 5 is configured on the inner wall of the housing and close to the light aperture. Further, the reflector 5 is positioned opposite to the photosensitive sensor 3 and adjacent to the light aperture, and a reflecting surface of the reflector is faced towards the photosensitive sensor 3.

In one example, the beam shaping lens 6 is used for transforming the light beam emitted by the laser light source 2 from an elliptical light beam to a circular beam.

In one example, the beam shaping lens 6 is provided with a beam input side and a beam output side.

As shown in FIGS. 4 and 5, a light beam emitted from the laser light source 2 has a divergence angle of 30 degree on the major-axis, and a divergence angle of 9 degree on the minor-axis when entering the beam input side of the beam shaping lens 6. The light beam emitted from the laser light source 2 has a divergence angle of 19.26 degree on the both major-axis and minor-axis when exiting the beam output side of the shaping lens 6.

The laser light source 2 is distanced from the beam input side of the beam shaping lens 6 with a distance of 2.55 mm, and the laser light source 2 is distanced from the beam input side of the beam shaping lens 6 with a distance of 6.55 mm.

In one example, the beam homogenizing lens 7 is an aspherical lens, configured for transforming the light beam that passes through the beam shaping lens into a flat-top distribution to obtain a circular collimated flat-top beam with a uniform intensity distribution.

The present disclosure further discloses a monitoring method for using in the laser energy monitor for myopia treatment device, and the method comprises the steps of:
Step 1. initiating a laser light source 2 and emitting a light beam;
Step 2. receiving a portion of the light beam from the laser light source 2 with a photosensitive sensor 3 and obtaining a light intensity, then transmitting the light intensity to a controller;
Step 3. adjusting a brightness of the laser light source according to the detected light intensity from the controller.

In one example, in Step 2, when the reflector 5 is configured on an inner wall of the housing 1, the light intensity of the light beam at the light aperture detected by a sensor is set as A, and the intensity of light beam detected by the photosensitive sensor 3 is set as B, and the light beam attenuation coefficient x is calculated by comparing B with A. Therefore, a light beam attenuation compensation formula is obtained as follows: A=B*x. According to the light intensity detected by the photosensitive sensor 3 and the light beam attenuation coefficient (x), the light intensity of the light beam at the light aperture is calculated by such a compensation formula in real time.

In conclusion, the monitor of the present disclosure has adjusted the inner layout of the conventional housing. Specifically, the beam shaping structure and the sensing structure are integrated inside the housing 1 to reduce the size of the monitor. After the irradiation intensity of laser light in a unit area is calculated, the light intensity of the laser light beam passing through the light aperture is finally obtained. Therefore, the light intensity of the light beam passing through the light aperture can be obtained without complicate and fine adjustment.

Further, laser light that passes through the light aperture are not blocked by the semi-transparent and semi-reflective mirror 4, thereby achieving a desirable light intensity with a lower power-consumption, compared with the conventional art.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A laser energy monitor for myopia treatment device, comprising:
a laser light source, a housing, a beam shaping lens, a beam homogenizing lens, a photosensitive sensor, and a controller;
wherein the housing is provided with a cavity for receiving the laser light source, the beam shaping lens, the beam homogenizing lens, and the photosensitive sensor, the housing is also provided with a light aperture, the light aperture is communicated to the cavity, and the laser light source is positioned opposite to the light aperture;
the beam shaping lens and the beam homogenizing lens are configured between the laser light source and the light aperture, and the light aperture, the beam homogenizing lens, the beam shaping lens, and the laser light source are successively arranged from top to bottom; and
the photosensitive sensor is electrically connected to the controller.

2. The laser energy monitor of claim 1, wherein the photosensitive sensor is configured on an inner wall of the housing and adjacent to the light aperture.

3. The laser energy monitor of claim 1, wherein the photosensitive sensor is configured on an inner wall of the housing and adjacent to the laser light source, and a reflector is configured on the inner wall of the housing and adjacent to the light aperture.

4. The laser energy monitor of claim 3, wherein the reflector is positioned opposite to the photosensitive sensor and adjacent to the light aperture, and a reflecting surface of the reflector is faced towards the photosensitive sensor.

5. The laser energy monitor of claim 1, wherein the beam shaping lens is used for transforming a light beam emitted by the laser light source from an elliptical beam to a circular beam.

6. The laser energy monitor of claim 5, wherein the beam shaping lens is provided with a beam input side and a beam output side;
the light beam emitted from the laser light source has a divergence angle from 10 to 45 degrees on the major-axis, and a divergence angle from 0 to 30 degrees on the minor-axis when entering the beam input side of the beam shaping lens, and the light beam emitted from the laser light source have a divergence angle from 0 to 30 degrees on both major-axis and minor-axis when exiting the beam output side of the beam shaping lens.

7. The laser energy monitor of claim 6, wherein the laser light source is distanced from the beam input side of the beam shaping lens in a range from 1 to 10 mm, and the laser light source is distanced from the beam output side of the beam shaping lens in a range from 5 to 20 mm.

8. The laser energy monitor of claim 1, wherein the beam homogenizing lens is an aspherical lens, configured for transforming the light beam that passes through the beam shaping lens into a flat-top distribution to obtain a circular collimated flat-top beam with a uniform intensity distribution.

9. A monitoring method for using in the laser energy monitor of claim 1, comprising the steps of:
Step 1. initiating a laser light source and emitting a light beam;
Step 2. receiving a portion of the light beam from the laser light source with a photosensitive sensor and obtaining a light intensity, then transmitting the light intensity to a controller;
Step 3. adjusting a brightness of the laser light source according to the detected light intensity from the controller.

10. The monitoring method of claim 9, wherein in the Step 2, when the reflector is configured on an inner wall of the housing, the light intensity of the light beam at the light aperture detected by a sensor is set as A, and the intensity of light beam detected by the photosensitive sensor is set as B, and the light beam attenuation coefficient x is calculated by comparing B with A; a light beam attenuation compensation formula is obtained as follows: A=B*x; and according to the light intensity detected by the photosensitive sensor and the light beam attenuation coefficient, the light intensity of the light beam at the light aperture is calculated by such a compensation formula in real time.
